SEQUENCE LISTING (10 sequences)

SEQ ID NO: 1 - DNA, 1173 bp, Homo sapiens
atgccagatactaatagcacaatcaatttatcactaagcactcgtgttactttagcattttttatgtccttagtagcttttgctataatgctaggaaatgctttggtcattttagcttttgtggtggacaaaaaccttagacatcgaagtagttatttttttcttaacttggccatctctgacttctttgtgggtgtgatctccattcctttgtacatccctcacacgctgttcgaatgggattttggaaaggaaatctgtgtattttggctcactactgactatctgttatgtacagcatctgtatataacattgtcctcatcagctatgatcgatacctgtcagtctcaaatgctgtgtcttatagaactcaacatactggggtcttgaagattgttactctgatggtggccgtttgggtgctggccttcttagtgaatgggccaatgattctagtttcagagtcttggaaggatgaaggtagtgaatgtgaacctggatttttttcggaatggtacatccttgccatcacatcattcttggaattcgtgatcccagtcatcttagtcgcttatttcaacatgaatatttattggagcctgtggaagcgtgatcatctcagtaggtgccaaagccatcctggactgactgctgtctcttccaacatctgtggacactcattcagaggtagactatcttcaaggagatctctttctgcatcgacagaagttcctgcatcctttcattcagagagacagaggagaaagagtagtctcatgttttcctcaagaaccaagatgaatagcaatacaattgcttccaaaatgggttccttctcccaatcagattctgtagctcttcaccaaagggaacatgttgaactgcttagagccaggagattagccaagtcactggccattctcttaggggtttttgctgtttgctgggctccatattctctgttcacaattgtcctttcattttattcctcagcaacaggtcctaaatcagtttggtatagaattgcattttggcttcagtggttcaattcctttgtcaatcctcttttgtatccattgtgtcacaagcgctttcaaaaggctttcttgaaaatattttgtataaaaaagcaacctctaccatcacaacacagtcggtcagtatcttcttaa

SEQ ID NO: 2 - protein, 390 aa, Homo sapiens
MPDTNSTINLSLSTRVTLAFFMSLVAFAIMLGNALVILAFVVDKNLRHRSSYFFLNLAISDFFVGVISIPLYIPHTLFEWDFGKEICVFWLTTDYLLCTASVYNIVLISYDRYLSVSNAVSYRTQHTGVLKIVTLMVAVWVLAFLVNGPMILVSESWKDEGSECEPGFFSEWYILAITSFLEFVIPVILVAYFNMNIYWSLWKRDHLSRCQSHPGLTAVSSNICGHSFRGRLSSRRSLSASTEVPASFHSERQRRKSSLMFSSRTKMNSNTIASKMGSFSQSDSVALHQREHVELLRARRLAKSLAILLGVFAVCWAPYSLFTIVLSFYSSATGPKSVWYRIAFWLQWFNSFVNPLLYPLCHKRFQKAFLKIFCIKKQPLPSQHSRSVSS

SEQ ID NO: 3 - DNA, 27 bp, Homo sapiens
accatgccagatactaatagcacaatc

SEQ ID NO: 4 - DNA, 24 bp, Homo sapiens
ttaagaagatactgaccgactgtg

SEQ ID NO: 5 - DNA, 25 bp, Homo sapiens
gggcaagataaagggcagaccagat

SEQ ID NO: 6 - DNA, 26 bp, Homo sapiens
tccttctcccaatcagattctgtagc

SEQ ID NO: 7 - DNA, 22 bp, Homo sapiens
gtatccattgtgtcacaagcgc

SEQ ID NO: 8 - DNA, 24 bp, Homo sapiens
aatattttattggagcctgtggaa

SEQ ID NO: 9 - DNA, 22 bp, Homo sapiens
ggaagagacagcagtcagtcca

SEQ ID NO: 10 - DNA, 29 bp, Homo sapiens
acacaaaggatatcagcgaaaggcaagcc

(19) United States
(12) Patent Application Publication
(10) Pub. No.: US 2002/0132755 A1
(43) Pub. Date: Sep. 19, 2002

(54) Histamine receptor antagonists

(75) Inventors: Stephen Jenkinson, Durham, NC (US); Mark Anthony O'Reilly, Sandwich (GB); Michael Andrew Trevethick, Sandwich (GB)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/052,193

(22) Filed: Jan. 17, 2002

Related U.S. Application Data
(60) Provisional application No. 60/270,641, filed on Feb. 22, 2001.

(30) Foreign Application Priority Data
Jan. 17, 2001 (GB) 0101223.6

(51) Int. Cl. A61K 31/00

(52) U.S. Cl. 514/1

(57) ABSTRACT

The present invention provides a combination of (a) a histamine H₁ receptor antagonist and (b) an antagonist that is at least 10-fold selective for the histamine H₄ receptor as compared to the histamine H₃ receptor; and an antagonist that is at least 10-fold selective for the histamine H₄ receptor as compared to the histamine H₃ receptor: and compositions and uses thereof.

This invention relates to a combination of a histamine H₁ receptor antagonist and a selective histamine H₄ receptor antagonist for the treatment of a range of diseases that may be treated by antagonism of either or both of the H₁ and H₄ receptors including allergic diseases and disorders such as allergic rhinitis and asthma, to the uses of, and to compositions, products and methods of treatment including, such a combination. This invention also relates to a selective histamine H₄ receptor antagonist.

Allergies are widespread and affect a large proportion of the world population. They may be seasonal in nature and can be caused by a variety of factors present in the environment such as pollen, mites and dust particles.

Symptoms of allergic rhinitis, which may be seasonal or perennial, can include rhinorrhea, nasal congestion and irritation, often accompanied by coughing or sneezing. Irritation and soreness of the throat and eyes is also common. The level of severity of each symptom experienced may vary from representing a minor problem to a person experiencing a severe effect.

The use of a histamine H₁ receptor antagonist in the treatment of allergic rhinitis is well-documented but when administered alone it does not provide an effective relief for nasal blockage and such agents are therefore often administered concurrently with sympathomimetic amine decongestants such as phenylpropanolamine and pseudoephedrine. However, such concommitant therapy is not suitable for all patients since central nervous system and cardiovascular side effects are often observed. WO98/06394 discloses the use of a combination of a histamine H₁ receptor antagonist and a histamine H₃ receptor antagonist for the treatment of allergy-induced responses in the mammalian airway, including relief from nasal congestion.

There exists a need for additional effective treatments of allergic diseases and disorders such as allergic rhinitis and asthma. There is a particular need for an alternative therapy to provide relief from most, if not all, the symptoms of allergic rhinitis, including nasal congestion.

The present invention is based on the teaching of EP 1096009 A1 (European Patent Application no. 00309364.8) and in co-pending U.S. non-provisional application Ser. No. 09/698,801 in which, inter alia, a polynucleotide encoding for a polypeptide corresponding to a G-protein coupled receptor (GPCR), newly termed by other researchers the histamine H₄ receptor, are described and claimed. Although a compound that antagonises or selectively antagonises such a polypeptide is also disclosed in general terms, there is no definition or discussion of the meaning of selectivity. Further, there is no mention that antagonists of such a polypeptide may be used in combination with a histamine H₁ receptor antagonist. The teaching of these documents is incorporated herein by reference in their entirety for all purposes. Specifically, in the document the histamine H₄ receptor is defined as a polypeptide comprising:

(a) a polypeptide having the deduced amino acid sequence translated from the polynucleotide sequence in SEQ ID NO: 1 and variants, fragments, homologues, analogues and derivatives thereof;

(b) a polypeptide of SEQ ID NO: 2 and variants, fragments, homologues, analogues and derivatives thereof; or

(c) a polypeptide encoded by the cDNA of NCIMB 41073 and variants, fragments, homologues, analogues and derivatives thereof.

The definitions of the above terms relating to the polypeptide are to be understood to be in conformity with the teaching of EP 1096009 A1 (European Patent Application no. 00309364.8 corresponding to U.S. Non-provisional application Ser. No. 09/698,801) and, in particular, the terms “variant”, “homologue”, “fragment”, “analogue” or “derivative” in relation to the amino acid sequence for the polypeptide include any substitution of, variation of, modification of, replacement of, deletion of or addition of one (or more) amino acid from or to the sequence providing the resultant polypeptide has GPCR activity, preferably being at least as biologically active as the polypeptide shown in attached SEQ ID NO: 2. In particular, the term “homologue” covers homology with respect to structure and/or function. With respect to sequence homology, there is at least 70%, preferably at least 75%, more preferably at least 80%, more preferably at least 85%, more preferably at least 90%, more preferably at least 95% homology to the sequence shown in SEQ ID NO: 2. Most preferably there is at least 98% homology to the sequence shown in SEQ ID NO: 2.

This polypeptide corresponds to the orphan G-protein-coupled receptor encoded GPRv53, that has been surmised to be a novel histamine H₄ receptor, as described in The Journal of Biological Chemistry, 275(47), 36781-36786 (November 2000), the teaching of which is incorporated herein by reference.

We have surprisingly found that histamine-induced chemotaxis (migration towards a chemoattractant) of human eosinophils appears to be mediated by the histamine H₄ receptor and not by the histamine H₃ receptor and therefore that histamine H₄ receptor antagonists prevent histamine-induced chemotaxis of human eosinophils. Eosinophils have been implicated in the pathogenesis of inflammatory and allergic diseases such as asthma and allergic rhinitis and high levels of histamine are released in patients with these conditions. Histamine H₄ receptor antagonists may be advantageously used in treating such conditions since (i) they are likely to inhibit eosinophil infiltration into the nose and thus relieve nasal congestion by an additional or independent mechanism to antagonism of the histamine H₃ receptor, or (ii) by inhibiting eosinophil infiltration into the lung they are likely to reduce the inflammation associated with asthma and provide an effective alternative treatment for this disease.

A combination of a selective histamine H₄ receptor antagonist and a histamine H₁ receptor antagonist may be advantageously used to treat allergic rhinitis since it would treat all the main symptoms of this disease including nasal congestion. A combination of a selective histamine H₄ receptor antagonist and a histamine H₁ receptor antagonist may be advantageously used to treat asthma since it may improve lung function in a synergistic manner.

The present invention provides a combination of (a) a histamine H₁ receptor antagonist and (b) an antagonist that is at least 10-fold selective for the histamine H₄ receptor as compared to the histamine H₃ receptor.

The present invention also provides a combination of (a) a histamine H₁ receptor antagonist and (b) an antagonist that is at least 10-fold selective for the histamine H₄ receptor as compared to the histamine H₃ receptor, for administration simultaneously, separately or sequentially, for use as a medicament for the treatment of a disease or disorder that may be treated by antagonism of either or both of the histamine H₁ and H₄ receptors such as allergic rhinitis or asthma.

The present invention further provides the use of a combination of (a) a histamine H₁ receptor antagonist and (b) an antagonist that is at least 10-fold selective for the histamine H₄ receptor as compared to the histamine H₃ receptor, for administration simultaneously, separately or sequentially, for the manufacture of a medicament for the treatment of a disease or disorder that may be treated by antagonism of either or both of the histamine H₁ and H₄ receptors such as allergic rhinitis or asthma.

The present invention further provides a method of treatment of a mammal, including a human being, to treat a disease or disorder that may be treated by antagonism of either or both of the histamine H₁ and H₄ receptors such as allergic rhinitis or asthma including simultaneous, separate or sequential administration to the mammal of a (a) an effective amount of a histamine H₁ receptor antagonist and (b) an effective amount of an antagonist that is at least 10-fold selective for the histamine H₄ receptor as compared to the histamine H₃ receptor.

The present invention further provides a pharmaceutical composition including (a) a histamine H₁ receptor antagonist and (b) an antagonist that is at least 10-fold selective for the histamine H₄ receptor as compared to the histamine H₃ receptor, and a pharmaceutically acceptable excipient, diluent or carrier.

The present invention further provides a product containing (a) a histamine H₁ receptor antagonist and (b) an antagonist that is at least 10-fold selective for the histamine H₄ receptor as compared to the histamine H₃ receptor as a combined preparation for simultaneous, separate or sequential use in the treatment of a disease or disorder that may be treated by antagonism of either or both of the histamine H₁ and H₄ receptors such as allergic rhinitis or asthma.

The present invention further provides an antagonist that is at least 10-fold selective for the histamine H₄ receptor as compared to the histamine H₃ receptor, the uses of and compositions and methods of treatment including, such an antagonist. Such a selective antagonist has not been previously described and thioperamide and clobenpropit, both of which are histamine H₃ receptor antagonists, do not display this degree of selectivity.

The definition “an antagonist that is at least 10-fold selective for the histamine H₄ receptor as compared to the histamine H₃ receptor” as used herein means an antagonist that is at least 10-fold selective for the histamine H₄ receptor as defined in EP 1096009 A1 (European Patent Application no. 00309364.8) or as the orphan G-protein-coupled receptor encoded GPRv53 as disclosed in The Journal of Biological Chemistry, 275(47), 36781-36786 (November 2000) as described above, as compared to the known histamine H₃ receptor, by the biological assays described below.

Preferably, the antagonist is at least 30-fold selective for the histamine H₄ receptor as compared to the histamine H₃ receptor.

Preferably, the antagonist is at least 50-fold selective for the histamine H₄ receptor as compared to the histamine H₃ receptor.

Preferably, the antagonist is at least 100-fold selective for the histamine H₄ receptor as compared to the histamine H₃ receptor.

Preferably, the antagonist is at least 1000-fold selective for the histamine H₄ receptor as compared to the histamine H₃ receptor.

The definition “histamine H₁ receptor antagonist” is well-understood in the art and is in accordance therewith.

The diseases or disorders that may be treated with the present combination or antagonist include those which may be modulated by antagonism of the H₄ receptor such as those which concern aspects of signal transduction. Useful therapeutic areas include, but are not limited to, obesity, diabetes and metabolic disease, neurological disease, psychotherapeutics, urogenital disease, reproduction and sexual medicine, inflammation, cancer, tissue repair, dermatology, skin pigmentation, photoageing, frailty, osteoporosis, cardiovascular disease, gastrointestinal disease, anti-infection, allergy and respiratory disease, sensory organ disorders, sleep disorders and hairloss.

Preferred diseases or disorders that may be treated are allergic disorders such as extrinsic asthma, rhinitis (allergic and chronic), onchocercal dermatitis, atopic dermatitis, drug reactions and NERDS (nodules, eosinophilia, rheumatism, dermatitis and swelling), vasculitic granulomatous diseases such as temporal vasculitis, Churg-Strauss syndrome, polyarteritis, Wegner's granulomatosis and eosinophilic granulomatous prostatitis, immunological disorders such as autoimmune reactions (e.g. multiple sclerosis), graft rejection and intrinsic asthma, interstitial and other pulmonary diseases such as chronic obstructive pulmonary disease (COPD), eosinophilic pleural effusions, transient pulmonary eosinophilic infiltrates (Loffler), histiocytosis, chronic eosinophilic pneumonia, hypersensitivity pneumonitis, allergic bronchopulmonary aspergillosis, sarcoidosis, idiopathic pulmonary fibrosis and topical eosinophilia, infectious parasitic diseases such as toxocariasis, filariasis, schistosomiasis, trichinosis, strongyloides, ascariasis and echinococcosis/cysticercosis, other infectious diseases such as acute coccidioidomycosis, cat scratch disease, afebrile tuberculosis and chlamydial pneumonia at infancy, neoplastic and myeloproliferative diseases such as bronchogenic carcinoma, hypereosinophilic syndrome, T-cell lymphomas and Hodgkin's disease, inflammatory conditions such as inflammatory bowel diseases (e.g. ulcerative colitis, Crohn's disease) and sinusitis, and coeliac disease, obstructive hepatic disease and dermatitis herpetiformis.

Particularly preferred diseases or disorders that may be treated are selected from the group consisting of asthma (both extrinsic and intrinsic), rhinitis (allergic and chronic), COPD and inflammatory bowel diseases, or that consisting of allergic disorders, vasculitic granulomatous diseases, immunological disorders, interstitial and other pulmonary diseases, infectious diseases, inflammatory diseases, and neoplastic and myeloproliferative diseases. Preferably, said allergic disorder is extrinsic asthma or rhinitis (allergic or chronic), said immunological disorder is intrinsic asthma, said pulmonary disease is COPD and said inflammatory diseases are inflammatory bowel diseases.

The selectivity of an antagonist for the histamine H₃ or H₄ receptor can be determined by the following procedures.

The cDNA for the human histamine H₃ and H₄ receptor can be expressed in a suitable cell line such as HEK293 or CHO. In addition membranes can be prepared from tissues or cells which naturally express either the histamine H₃ and/or H₄ receptor. Affinity can be assessed at either receptor by assessing the ability of ligands to inhibit the binding of a suitable radioactively labelled ligand to membranes containing either the histamine H₃ or H₄ receptor. The potency of compounds is compared by calculating their pKᵢ values.

The functional activity of ligands to determine agonist or antagonist activity is determined in whole cells expressing either the H₃ or H₄ receptor.

Agonists (full or partial) are identified as agents which inhibit forskolin stimulated cyclic AMP production which can be measured directly (as cyclic AMP) or indirectly as changes in beta-lactamase activity in cells co-expressing the CRE-beta-lactamase reporter system and the appropriate histamine receptor. Agonist activities may be expressed as IC₅₀ values. Inverse agonists can also be identified by the inhibition of the basal cAMP activity (i.e. in the absence of forskolin). Agents which have no effect on forskolin stimulated cAMP or inhibit basal cAMP may be antagonists and such activity can be measured in an assay where cells are pre-incubated with the ligand of interest and their ability to inhibit H₃ or H₄ agonist-induced reduction of forskolin stimulated cyclic AMP is measured as described above. The experiments can be performed in one of two ways. Firstly, by increasing the concentration of antagonist versus fixed concentration of agonist and calculating the IC₅₀ for each antagonist. Secondly, by increasing the concentration of antagonist versus increasing the concentration of agonist and expressing the antagonist potency as a pk_{b} or pA₂ value, as appropriate.

A further functional assay for the histamine H₄ receptor determines the ability of histamine to induce chemotaxis in eosinophils isolated from human blood. In this assay cells are primed with interleukin 5 and the ability of histaminergic ligands to promote migration of eosinophils across a permeable membrane is studied. The number of cells migrating in a defined period is then counted. Agonist potencies are expressed as EC₅₀ values. Antagonists can also be studied by pre-incubating eosinophils with the compound and then assessing the inhibitory effects on histamine (or other suitable ligand) on eosinophil chemotaxis. The experiments can be performed in one of two ways. Firstly, by increasing the concentration of antagonist versus fixed concentration of agonist and calculating the IC₅₀ for each antagonist. Secondly, by increasing the concentration of antagonist versus increasing the concentration of agonist and expressing the antagonist potency as a pk_{b} or pA₂ value, as appropriate. Using this assay we have shown that histamine-induced chemotaxis of human eosinophils appears to be mediated by the histamine H₄ receptor and not by the histamine H₃ receptor.

The present combination (or each active element thereof) or antagonist can be administered alone but will generally be administered in admixture with a suitable pharmaceutical excipient, diluent or carrier selected with regard to the intended route of administration and standard pharmaceutical practice.

For example, the present combination or antagonist can be administered orally, buccally or sublingually in the form of tablets, capsules, multi-particulates, gels, films, ovules, elixirs, solutions or suspensions, which may contain flavouring or colouring agents, for immediate-, delayed-, modified-, sustained-, pulsed- or controlled-release applications. The present combination or antagonist may also be administered as fast-dispersing or fast-dissolving dosage forms or in the form of a high energy dispersion or as coated particles. Suitable formulations of the present combination or antagonist may be in coated or uncoated form, as desired.

Such solid pharmaceutical compositions, for example, tablets, may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

### GENERAL EXAMPLE

A formulation of a tablet could typically contain from 0.01 mg to 500 mg of each active element of the combination or of the antagonist whilst tablet fill weights may range from 50 mg to 1000 mg. An example of a formulation for a 10 mg tablet is illustrated below:

| Ingredient | % w/w |
|---|---|
| Pharmaceutically active agent(s) | 10.000* |
| Lactose | 64.125 |
| Starch | 21.375 |
| Croscarmellose sodium | 3.000 |
| Magnesium Stearate | 1.500 |

*Quantity adjusted in accordance with drug activity.

The tablets are manufactured by a standard process, for example, direct compression or a wet or dry granulation process. The tablet cores may be coated with appropriate overcoats.

Solid compositions of a similar type may also be employed as fillers in gelatin or HPMC capsules. Preferred excipients in this regard include lactose, starch, a cellulose, milk sugar or high molecular weight polyethylene glycols. For aqueous suspensions and/or elixirs, the present combination or antagonist may be combined with various sweetening or flavouring agents, colouring matter or dyes, with emulsifying and/or suspending agents and with diluents such as water, ethanol, propylene glycol and glycerin, and combinations thereof.

The present combination or antagonist can also be administered parenterally, for example, intravenously, intra-arterially, intraperitoneally, intrathecally, intraventricularly, intraurethrally, intrasternally, intracranially, intramuscularly or subcutaneously, or they may be administered by infusion or needleless injection techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

For oral and parenteral administration to human patients, the daily dosage level of each active element of the present combination or of the present antagonist will usually be from 0.01 to 50 mg/kg body weight of the subject to be treated, preferably from 0.1 to 20 mg/kg (in single or divided doses), or they may be administered by intravenous infusion at a dose of from 0.001 to 10 mg/kg/hour.

For oral, parenteral, buccal or sublingual administration to a subject to be treated, the daily dosage level of each active element of the combination or of the antagonist may typically be from 10 to 500 mg (in single or divided doses). Thus tablets or capsules of the present combination or antagonist may contain from 5 to 100 mg of each active element of the combination or of the antagonist for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The present combination or antagonist can also be administered intranasally or by inhalation and are conveniently delivered in the form of a dry powder inhaler or an aerosol spray presentation from a pressurised container, pump, spray, atomiser or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray, atomiser or nebuliser may contain a solution or suspension of the active compound(s), e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the present combination or antagonist and a suitable powder base such as lactose or starch.

Aerosol or dry powder formulations are preferably arranged so that each metered dose or “puff” contains from 10 μg to 1 mg of each active element of the present combination or antagonist for delivery to the patient. The overall daily dose with an aerosol will be in the range of from 10 μg to 10 mg of each active element of the present combination or antagonist which may be administered in a single dose or, more usually, in divided doses throughout the day.

Alternatively, the present combination or antagonist can be administered in the form of a suppository or pessary, or they may be applied topically in the form of a gel, hydrogel, lotion, solution, cream, ointment or dusting powder. The present combination or antagonist may also be dermally or transdermally administered, for example, by the use of a skin patch. They may also be administered by the pulmonary or rectal routes.

They may also be administered by the ocular route, particularly for treating allergic conditions of the eye. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the present combination or antagonist can be formulated as a suitable ointment containing the active compound(s) suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Each element of the present combination or the present antagonist may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

The present invention embraces any other suitable formulations and administration routes described in EP 1096009 A1 (European Patent Application no. 00309364.8).

The present combination or antagonist can also be administered together with a steroid, a beta-adrenoceptor agonist, a muscarinic antagonist or a mucolytic.

The present combination, antagonist or composition can be used to treat the above-mentioned conditions in humans or in other animals.

It is to be appreciated that all references herein to treatment include curative, palliative and prophylactic treatment.

### PHARMACOLOGICAL DATA

a) Selectivity of antagonists for the histamine H₃ and H₄ receptors

WO98/06394 discloses histamine H₃ receptor antagonists including thioperamide and clobenpropit without mention of any antagonist activity for the histamine H₄ receptor.

Thioperamide, clobenpropit and iodophenpropit were tested for (i) binding affinity for the histamine H₄ receptor using the method described on page 7 using the HEK293 cell line, and (ii) binding affinity for the histamine H₃ receptor using the method described on page 7 using human brain tissue which naturally expresses the histamine H₃ receptor. The results are shown below.

| Compound | H₄ binding affinity (pKi) | H₃ binding affinity (pKi) |
|---|---|---|
| Thioperamide | 7.26 +/− 0.06 | 6.79 +/− 0.10 |
| Clobenpropit | 8.18 +/− 0.02 | 8.63 +/− 0.04 |
| Iodophenpropit | 7.87 +/− 0.04 | 7.94 +/− 0.10 |

(A difference of 1.0 log. unit would indicate a 10-fold selectivity for the receptor for which the highest pKi value was obtained)

These data clearly show that thioperamide, clobenpropit and iodophenpropit are both histamine H₃ and H₄ receptor antagonists but have little selectivity for the histamine H₄ receptor over the histamine H₃ receptor. They are certainly not 10-fold selective.

b) Histamine-induced chemotaxis of human eosinophils

Using the interleukin 5 priming method of page 8 it was demonstrated that histamine promoted chemotaxis of human isolated eosinophils over a concentration range of 10⁻⁸ to 10⁻⁵ M. However, agonists individually selective for the H₁ (HTMT), H₂ (dimaprit) and H₃ (imetit) receptors each did not promote chemotaxis of human eosinophils at concentrations of from 10⁻⁸ to 10⁻⁵ M.

Using the eosinophil pre-incubation method of page 8 it was demonstrated that the non-selective H₄/H₃ antagonists thioperamide maleate and clobenpropit dihydrobromide inhibited histamine-induced chemotaxis of human eosinophils in a concentration related manner with IC₅₀s of 0.83 and 1.50 micromolar, respectively. In contrast antagonists individually selective for the H₁ receptor (mepyramine maleate) and the H₂ receptor (cimetidine) had little or no effect on histamine-induced chemotaxis at concentrations of 10 micromolar.

These data show that histamine-induced chemotaxis of human eosinophils must be mediated by activation of the histamine H₄ receptor. As such histamine H₄ receptor antagonists may be advantageously used in treating asthma and allergic rhinitis since (i) they are likely to inhibit eosinophil infiltration into the nose and thus relieve nasal congestion by an additional or independent mechanism to antagonism of the histamine H₃ receptor, or (ii) by inhibiting eosinophil infiltration into the lung they are likely to reduce the inflammation associated with asthma and provide an effective alternative treatment for this disease. A combination of a selective histamine H₄ receptor antagonist and a histamine H₁ receptor antagonist may therefore be advantageously used to treat allergic rhinitis since it would treat all the main symptoms of this disease including nasal congestion. A combination of a selective histamine H₄ receptor antagonist and a histamine H₁ receptor antagonist may also be advantageously used to treat asthma since it may improve lung function in a synergistic manner.

1. A combination of (a) a histamine H₁ receptor antagonist and (b) an antagonist that is at least 10-fold selective for the histamine H₄ receptor as compared to the histamine H₃ receptor.

2. A combination of (a) a histamine H₁ receptor antagonist and (b) an antagonist that is at least 10-fold selective for the histamine H₄ receptor as compared to the histamine H₃ receptor, for use as a medicament.

3. A combination of (a) a histamine H₁ receptor antagonist and (b) an antagonist that is at least 10-fold selective for the histamine H₄ receptor as compared to the histamine H₃ receptor, for administration simultaneously, separately or sequentially, for use as a medicament for the treatment of a disease or disorder that may be treated by antagonism of either or both of the histamine H₁ and H₄ receptors such as allergic rhinitis or asthma.

4. The use of a combination of (a) a histamine H₁ receptor antagonist and (b) an antagonist that is at least 10-fold selective for the histamine H₄ receptor as compared to the histamine H₃ receptor, for administration simultaneously, separately or sequentially, for the manufacture of a medicament for the treatment of a disease or disorder that may be treated by antagonism of either or both of the histamine H₁ and H₄ receptors such as allergic rhinitis or asthma.

5. A method of treatment of a mammal, including a human being, to treat a disease or disorder that may be treated by antagonism of either or both of the histamine H₁ and H₄ receptors such as allergic rhinitis or asthma including simultaneous, separate or sequential administration to the mammal of a (a) an effective amount of a histamine H₁ receptor antagonist and (b) an effective amount of an antagonist that is at least 10-fold selective for the histamine H₄ receptor as compared to the histamine H₃ receptor.

6. A pharmaceutical composition including (a) a histamine H₁ receptor antagonist and (b) an antagonist that is at least 10-fold selective for the histamine H₄ receptor as compared to the histamine H₃ receptor, and a pharmaceutically acceptable excipient, diluent or carrier.

7. A product containing (a) a histamine H₁ receptor antagonist and (b) an antagonist that is at least 10-fold selective for the histamine H₄ receptor as compared to the histamine H₃ receptor as a combined preparation for simultaneous, separate or sequential use in the treatment of a disease or disorder that may be treated by antagonism of either or both of the histamine H₁ and H₄ receptors such as allergic rhinitis or asthma.

8. An antagonist that is at least 10-fold selective for the histamine H₄ receptor as compared to the histamine H₃ receptor.

9. A pharmaceutical composition including an antagonist as claimed in claim 8 and a pharmaceutically acceptable excipient, diluent or carrier.

10. An antagonist as claimed in claim 8 or composition as claimed in claim 9 for use as a medicament.

11. An antagonist as claimed in claim 8 or composition as claimed in claim 9 for use as a medicament for the treatment of a disease or disorder that may be treated by antagonism of the histamine H₄ receptor such as allergic rhinitis or asthma.

12. The use of an antagonist as claimed in claim 8 or a composition as claimed in claim 9 for the manufacture of a medicament for the treatment of a disease or disorder that may be treated by antagonism of the histamine H₄ receptor such as allergic rhinitis or asthma.

13. A method of treatment of a mammal, including a human being, to treat a disease or disorder that may be treated by antagonism of the histamine H₄ receptor such as allergic rhinitis or asthma which includes administration to the mammal of an effective amount of an antagonist as claimed in claim 8 or a composition as claimed in claim 9.
